(19) United States
(12) Patent Application Publication
(10) Pub. No.: US 2023/0330636 A1
(43) Pub. Date: Oct. 19, 2023

(54) METHOD FOR PRODUCING ETHYL ACETATE PRODUCTION CATALYST

(71) Applicant: Resonac Corporation, Tokyo (JP)

(72) Inventors: Takuro SASAKI, Oita-shi (JP); Shintaro ITAGAKI, Oita-shi (JP); Yasuhiro HOSAGI, Oita-shi (JP); Yasuhiro IWAMA, Oita-shi (JP)

(73) Assignee: Resonac Corporation, Tokyo (JP)

(21) Appl. No.: 18/024,429

(22) PCT Filed: Aug. 23, 2021
(86) PCT No.: PCT/JP2021/030845

§ 371 Date: Mar. 2, 2023

(30) Foreign Application Priority Data
Sep. 29, 2020 (JP) 2020-163340

(51) Int. Cl. B01J 23/30; B01J 21/08; B01J 37/02; C07C 67/04

(52) U.S. Cl. CPC B01J 23/30; B01J 21/08; B01J 37/0201; B01J 37/0236; C07C 67/04

(57) ABSTRACT

Provided is a method for producing an ethyl acetate production catalyst that has high producibility and exceptional catalytic performance, the catalyst being such that a heteropolyacid and/or a salt thereof is carried near the surface of a carrier. A method for producing an ethyl acetate production catalyst, the method including, in the stated order: (1) an impregnation step in which a silica carrier is impregnated with an aqueous solution of a heteropolyacid or a salt thereof, constituting 80-105 vol % of the saturation absorption capacity of the carrier, to form an impregnated body; and (2) a drying step in which the impregnated body is dried at a fixed-rate drying speed of 5-300 g_{H2O}/kg_{supcat}·min.

### FIELD

The present invention relates to a method for producing an ethyl acetate production catalyst and a method for producing ethyl acetate using this catalyst.

### BACKGROUND

It is well known that from lower aliphatic carboxylic acids and lower olefins, corresponding esters can be produced via gas phase catalytic reactions. Furthermore, it is well known that supported catalysts in which a heteropolyacid and/or a salt thereof is supported on a carrier are useful (Patent Literature 1 to 3).

In gas-phase catalytic reactions using a supported catalyst, as a method for improving the performance of the catalyst, supporting an active ingredient near the surface of the carrier to increase the contact efficiency between the active ingredient and a reactant is known (Patent Literature 4 and 5).

For example, Patent Literature 4 describes that a catalyst in which an active ingredient is supported near the surface of a carrier can be obtained by impregnating the carrier with a solution in which the active ingredient is dissolved in an acetic acid solvent of 10 to 40% by volume of the water absorption amount of the carrier. Patent Literature 5 describes that a catalyst in which an active ingredient is supported near the surface of a carrier can be obtained by impregnating the carrier with a solution in which the active ingredient is dissolved in water of 10 to 70% by volume of the water absorption amount of the carrier, and drying the resulting impregnated body under reduced pressure at a predetermined rate.

However, in Patent Literature 4, acetic acid, which is used as the solvent, is harmful, and in Patent Literature 5, the method for drying the impregnated body is the vacuum drying method, and thus, neither of these production methods are suitable for industrial production of a catalyst. Furthermore, in these production methods, since the amount of solution impregnated in the carrier must be relatively small, such as 10 to 40% by volume or 10 to 70% by volume of the water absorption amount of the carrier, there is a risk that catalyst particles supporting a large amount of active ingredient and catalyst particles supporting little or no active ingredient may be generated.

### CITATION LIST

### Patent Literature

[PTL 1] JP H09-118647 A
[PTL 2] JP 2000-342980 A
[PTL 3] JP 2008-513534 A
[PTL 4] JP 2004-209469 A
[PTL 5] JP 2019-162604 A

### SUMMARY

### Technical Problem

In order to efficiently produce an ester from a lower aliphatic carboxylic acid and a lower olefin via a gas-phase catalytic reaction, it is necessary to produce a catalyst in which a heteropolyacid and/or a salt thereof is supported near the surface of a carrier. However, in a production method in which the amount of the impregnation solution used is maintained at a low level, it is difficult to control variations in the supported amount of the active ingredient between catalyst particles, and thus, a simple and industrial method for producing a catalyst having excellent activity and selectivity is desired.

In light of these circumstances, the present invention aims to provide a method for producing an ethyl acetate production catalyst having high productivity and excellent catalyst performance, in which a heteropolyacid and/or a salt thereof is supported near the surface of a carrier.

### Solution to Problem

As a result of rigorous investigation of methods for producing an ethyl acetate production catalyst containing a heteropolyacid and/or a salt thereof as an active ingredient, the present inventors have discovered that even when an aqueous solution of a heteropolyacid and/or a salt thereof (also simply referred to as an “aqueous heteropolyacid solution” in the present disclosure) having a volume close to 100% of the saturated water absorption capacity of a carrier is used as an impregnation solution, and the impregnation solution is uniformly impregnated into the inside of the carrier, in the drying step of the impregnated body, by setting the constant drying rate to within a large specific range, a large amount of active ingredient can be supported on the carrier surface, whereby an ethyl acetate production catalyst having high catalyst activity and excellent selectivity can be produced, and have completed the present invention.

Specifically, the present invention relates to the following [1] to [7].

[1]

A method for producing an ethyl acetate production catalyst, comprising, in this order:

(1) an impregnation step of impregnating a silica carrier with an aqueous solution of a heteropolyacid or a salt thereof in an amount of 80 to 105% by volume of the saturated water absorption capacity of the carrier to form an impregnated body, and

(2) a drying step of drying the impregnated body at a constant drying rate of 5 to 300 g_{H2O}/kg_{supcat}·min.

[2]

The method for producing an ethyl acetate production catalyst according to [1], wherein the constant drying rate in the drying step is 10 to 150 g_{H2O}/kg_{supcat}·min.

[3]

The method for producing an ethyl acetate production catalyst according to [1] or [2], wherein the constant drying rate in the drying step is 15 to 50 g_{H2O}/kg_{supcat}·min.

[4]

The method for producing an ethyl acetate production catalyst according to any one of [1] to [3], wherein a temperature of a drying medium used in the drying step is 80 to 130° C.

[5]

The method for producing an ethyl acetate production catalyst according any one of [1] to [4], wherein a drying medium in the drying step is air having a relative humidity of 0 to 60% RH, and the air is brought into contact with the impregnated body as an air flow to dry the impregnated body.

[6]

The method for producing an ethyl acetate production catalyst according to any one of [1] to [5], wherein a pressure in the drying step is normal pressure.

[7]

A method for producing ethyl acetate using ethylene and acetic acid as raw materials, wherein a reaction is carried out in the presence of an ethyl acetate production catalyst produced by the method according to any one of [1] to [6].

### Advantageous Effects of Invention

According to the present invention, an ethyl acetate production catalyst in which an active ingredient is present near the surface of a carrier and which exhibits high catalytic performance can be provided with high productivity.

### BRIEF DESCRIPTION OF DRAWINGS

FIG. 1 is an explanatory diagram of constant rate drying period.

FIG. 2 is an EPMA image of a catalyst in which the heteropolyacid of Example 1 is supported on a silica carrier.

FIG. 3 is an EPMA image of a catalyst in which the heteropolyacid of Comparative Example 1 is supported on a silica carrier.

FIG. 4 is a graph showing the by-product butene selectivity of each of Examples 1 to 5 and Comparative Examples 1 to 3.

### DESCRIPTION OF EMBODIMENTS

The preferred embodiments of the present invention will be described below, but the present invention is not limited to these embodiments, and it should be understood that various applications are possible within the scope of the spirit and execution thereof.

### [Production of Ethyl Acetate Production Catalyst]

In an embodiment, ethyl acetate is produced by reacting ethylene and acetic acid in a gas phase using a solid acid catalyst. The solid acid catalyst for the ethyl acetate production is a heteropolyacid or a salt thereof (also referred to as a “heteropolyacid salt” in the present disclosure), which is supported on a silica carrier in use.

### [Heteropolyacids and Salts]

A heteropolyacid is composed of a central element and peripheral elements bonded to oxygen. The central element is conventionally silicon or phosphorus, but may be any one selected from a variety of elements from Groups 1 to 17 of the periodic table of the elements. Specific examples thereof include, but are not limited to, a cupric ion; a divalent beryllium, zinc, cobalt or nickel ion; a trivalent boron, aluminum, gallium, iron, cerium, arsenic, antimony, phosphorus, bismuth, chromium or rhodium ion; a tetravalent silicon, germanium, tin, titanium, zirconium, vanadium, sulfur, tellurium, manganese, nickel, platinum, thorium, hafnium, cerium or another rare earth ion; a pentavalent phosphorus, arsenic, vanadium, or antimony ion; a hexavalent tellurium ion; and a heptavalent iodine ion. Specific examples of peripheral elements include, but are not limited to, tungsten, molybdenum, vanadium, niobium, and tantalum.

Such heteropolyacids are also known as “polyoxoanions”, “polyoxometalates”, or “metal oxide clusters.” Several well-known anion structures are named after researchers in the field, and for example, the Keggin-type structure, Wells-Dawson-type structure, and Anderson-Evans-Perloff-type structure are known. The details are described in “Chemistry of Polyacids” (Edited by The Chemical Society of Japan, Quarterly Chemical Review, No. 20, 1993). Heteropolyacids conventionally have a high molecular weight, for example, in the range of 700 to 8,500, and include not only a monomer but also dimeric complexes.

The heteropolyacid salt is not particularly limited as long as it is a metal salt or an onium salt in which some or all of the hydrogen atoms of a heteropolyacid described above are substituted. Specific examples include, but are not limited to, metal salts of lithium, sodium, potassium, cesium, magnesium, barium, copper, gold and gallium, and onium salts of ammonia, etc.

Particularly preferred examples of the heteropolyacid which can be used as a catalyst include, but are not limited to:

| | |
|---|---|
| Silicotungstic acid | H₄[SiW₁₂O₄₀]•xH₂O; |
| Phosphotungstic acid | H₃[PW₁₂O₄₀]•xH₂O; |
| Phosphomolybdic acid | H₃[PMo₁₂O₄₀]•xH₂O; |
| Silicomolybdic acid | H₄[SiMo₁₂O₄₀]•xH₂O; |
| Silicovanadotungstic acid | H₄₊ₙ[SiVₙW₁₂₋ₙO₄₀]•xH₂O; |
| Phosphovanadotungstic acid | H₃₊ₙ[PVₙW₁₂₋ₙO₄₀]•xH₂O; |
| Phosphovanadomolybdic acid | H₃₊ₙ[PVₙMo₁₂₋ₙO₄₀]•xH₂O; |
| Silicovanadomolybdic acid | H₄₊ₙ[SiVₙMo₁₂₋ₙO₄₀]•xH₂O; |
| Silicomolybdotungstic acid | H₄[SiMoₙW₁₂₋ₙO₄₀]•xH₂O; and |
| Phosphomolybdotungstic acid | H₃[PMoₙW₁₂₋ₙO₄₀]•xH₂O; |

wherein n is an integer of 1 to 11, and x is an integer of 1 or more.

The heteropolyacid is preferably silicotungstic acid, phosphotungstic acid, phosphomolybdic acid, silicomolybdic acid, silicovanadotungstic acid, or phosphovanadotungstic acid, and is more preferably silicotungstic acid or phosphotungstic acid.

The method for synthesizing such a heteropolyacid is not particularly limited, and any method may be used. For example, a heteropolyacid can be obtained by beating an acidic aqueous solution (approximately pH 1 to pH 2) containing a salt of molybdic acid or tungstic acid and a simple oxoacid of a heteroatom or a salt thereof. The heteropolyacid compound can be isolated, for example, by crystallization separation as a metal salt from the produced aqueous heteropolyacid solution. A specific example of the production of heteropolyacid is described on page 1413 of “New Experimental Chemistry 8, Synthesis of Inorganic Compound (III)” (edited by The Chemical Society of Japan, published by Maruzen Co., Ltd., Aug. 20, 1984, 3rd edition), but the production method is not limited to this. The structure of the synthesized heteropolyacid can be confirmed by X-ray diffraction, UV, or IR measurement as well as chemical analysis.

Preferred examples of heteropolyacid salts include lithium, sodium, potassium, cesium, magnesium, barium, copper, gold, gallium and ammonium salts of the preferred heteropolyacids described above.

Specific examples of heteropolyacid salts include a lithium salt of silicotungstic acid, a sodium salt of silicotungstic acid, a cesium salt of silicotungstic acid, a copper salt of silicotungstic acid, a gold salt of silicotungstic acid, and a gallium salt of silicotungstic acid; a lithium salt of phosphotungstic acid, a sodium salt of phosphotungstic acid, a cesium salt of phosphotungstic acid, a copper salt of phosphotungstic acid, a gold salt of phosphotungstic acid, and a gallium salt of phosphotungstic acid; a lithium salt of phosphomolybdic acid, a sodium salt of phosphomolybdic acid, a cesium salt of phosphomolybdic acid, a copper salt of phosphomolybdic acid, a gold salt of phosphomolybdic acid, and a gallium salt of phosphomolybdic acid; a lithium salt of silicomolybdic acid, a sodium salt of silicomolybdic acid, a cesium salt of silicomolybdic acid, a copper salt of silicomolybdic acid, a gold salt of silicomolybdic acid, and a gallium salt of silicomolybdic acid; a lithium salt of silicovanadotungstic acid, a sodium salt of silicovanadotungstic acid, a cesium salt of silicovanadotungstic acid, a copper salt of silicovanadotungstic acid, a gold salt of silicovanadotungstic acid, and a gallium salt of silicovanadotungstic acid; a lithium salt of phosphovanadotungstic acid, a sodium salt of phosphovanadotungstic acid, a cesium salt of phosphovanadotungstic acid, a copper salt of phosphovanadotungstic acid, a gold salt of phosphovanadotungstic acid, and a gallium salt of phosphovanadotungstic acid; a lithium salt of phosphovanadomolybdic acid, a sodium salt of phosphovanadomolybdic acid, a cesium salt of phosphovanadomolybdic acid, a copper salt of phosphovanadomolybdic acid, a gold salt of phosphovanadomolybdic acid, and a gallium salt of phosphovanadomolybdic acid; a lithium salt of silicovanadomolybdic acid, a sodium salt of silicovanadomolybdic acid, a cesium salt of silicovanadomolybdic acid, a copper salt of silicovanadomolybdic acid, a gold salt of silicovanadomolybdic acid, and a gallium salt of silicovanadomolybdic acid.

The heteropolyacid salt is preferably a lithium salt of silicotungstic acid, a sodium salt of silicotungstic acid, a cesium salt of silicotungstic acid, a copper salt of silicotungstic acid, a gold salt of silicotungstic acid, or a gallium salt of silicotungstic acid; a lithium salt of phosphotungstic acid, a sodium salt of phosphotungstic acid, a cesium salt of phosphotungstic acid, a copper salt of phosphotungstic acid, a gold salt of phosphotungstic acid, or a gallium salt of phosphotungstic acid; a lithium salt of phosphomolybdic acid, a sodium salt of phosphomolybdic acid, a cesium salt of phosphomolybdic acid, a copper salt of phosphomolybdic acid, a gold salt of phosphomolybdic acid, or a gallium salt of phosphomolybdic acid; a lithium salt of silicomolybdic acid, a sodium salt of silicomolybdic acid, a cesium salt of silicomolybdic acid, a copper salt of silicomolybdic acid, a gold salt of silicomolybdic acid, or a gallium salt of silicomolybdic acid; a lithium salt of silicovanadotungstic acid, a sodium salt of silicovanadotungstic acid, a cesium salt of silicovanadotungstic acid, a copper salt of silicovanadotungstic acid, a gold salt of silicovanadotungstic acid, or a gallium salt of silicovanadotungstic acid; a lithium salt of phosphovanadotungstic acid, a sodium salt of phosphovanadotungstic acid, a cesium salt of phosphovanadotungstic acid, a copper salt of phosphovanadotungstic acid, a gold salt of phosphovanadotungstic acid, or a gallium salt of phosphovanadotungstic acid.

As the heteropolyacid salt, it is particularly preferred to use the lithium salt of silicotungstic acid or the cesium salt of phosphotungstic acid.

### [Silica Carrier]

The silica carrier may have any shape, and the shape is not particularly limited, but it is preferably spherical or pellet-like. The particle size of the silica carrier varies depending on the form of the reaction, but when used in a fixed-bed system, it is preferably 2 mm to 10 mm, and more preferably 3 mm to 7 mm.

In an embodiment, supporting the heteropolyacid or salt thereof on a silica carrier includes, in this order, a step (impregnation step) of impregnating a silica carrier with an aqueous solution of a heteropolyacid or a salt thereof (aqueous heteropolyacid solution) at a specific impregnation ratio; and a step (drying step) of drying the carrier impregnated with the aqueous heteropolyacid solution under specific drying conditions. Between the impregnation step and the drying step, other steps (for example, an air-drying step, a transfer step from the impregnate device to the drying device, etc.) may be included, but these two steps are preferably performed continuously.

### [Impregnation Step]

In the impregnation step, for example, a spherical or pellet-shaped silica carrier absorbs the aqueous heteropolyacid solution as an impregnation liquid to form an impregnated body. It is preferable to agitate the carrier during the impregnation operation. The concentration of the heteropolyacid or salt thereof in the aqueous heteropolyacid solution is determined from the volume of the aqueous heteropolyacid solution calculated from the impregnation ratio and the amount of catalyst to be supported on the carrier. The concentration of the heteropolyacid or salt thereof in the aqueous heteropolyacid solution can generally be 0.8 to 1.2 kg/L.

The volume of the aqueous heteropolyacid solution impregnated in the carrier is in the range of 80 to 105% by volume, preferably in the range of 90 to 100% by volume, and more preferably in the range of 95 to 100% by volume, of the saturated water absorption capacity of the carrier. When the volume of the aqueous heteropolyacid solution is less than 80% by volume, there is a risk of mixing of catalyst particles on which the heteropolyacid or salt thereof is not supported. When the volume of the aqueous heteropolyacid solution is more than 105% by volume, the heteropolyacid or salt thereof which is not absorbed by the carrier is present in a free state, and there is a risk that the necessary amount of catalyst will not be uniformly supported on the carrier.

The “saturated water absorption capacity of the carrier” is the volume (L) of water which can be absorbed by a carrier having an apparent volume of 1 L. Details of the measurement method will be described later. The “impregnation ratio” is the volume ratio (% by volume) of the aqueous heteropolyacid solution absorbed by the carrier to the saturated water absorption capacity of the carrier, as illustrated by the following formula. The saturated water absorption capacity (L) and the volume (L) of the aqueous heteropolyacid solution are values at room temperature (23° C.).

Impregnation ratio (%)=100×volume of aqueous heteropolyacid solution absorbed by carrier having apparent volume of 1 L/saturated water absorption capacity of carrier

### [Drying Step]

In the drying step, the impregnated body is dried under specific drying conditions. Specifically, the drying rate (constant drying rate) during the constant rate drying period in the early stage of drying of the impregnated body is controlled within a specific range. The drying rate after the constant rate drying period may vary.

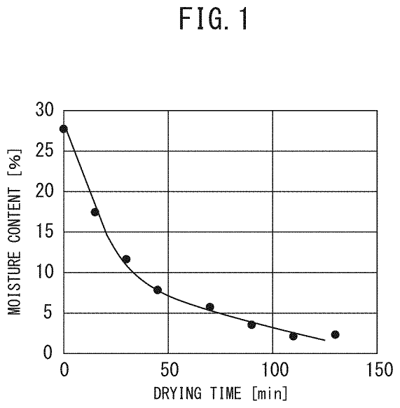

When the wet material is dried, the amount of decrease in moisture content per unit time (decrease rate) is constant in the early stage of drying (indicated linearly in the graph of drying time vs. moisture content), and then gradually reduces in the latter stages of drying. At this time, in the graph of drying time vs. moisture content, the section where the moisture content changes linearly is referred to as a “constant rate drying period”, and the drying rate during this period is referred to as a “constant drying rate.” The constant rate drying period depends on the structure of the drying device, the quantity of the object to be dried, the flow rate of the drying medium, temperature, and humidity. The constant rate drying period is preferably defined as 20 minutes after the start of drying, more preferably 15 minutes after the start of drying. It is most preferable to conduct a preliminary experiment of drying using actual devices and conditions in advance, create a graph as shown in FIG. 1, and determine the constant rate drying period. FIG. 1 is a graph showing the moisture content at each drying time when a silica carrier is impregnated with water (impregnation ratio 95%) and the silica carrier is air-dried at a temperature of 100° C. and an air flow speed of 13 m/min. In FIG. 1, the constant rate drying period is approximately 20 minutes from the start of drying. The constant drying rate is defined as a value obtained by dividing the difference (variation) between the amount of water contained in the impregnated body before drying and the amount of water contained in the impregnated body after drying for a predetermined time within the constant rate drying period (15 minutes from the start of drying in Example 1) by the drying time and the mass of the supported catalyst. The mass of the supported catalyst is the sum of the masses of the carrier and the anhydride of the heteropolyacid or salt thereof (the heteropolyacid or salt thereof excluding hydration water).

A specific calculation method of the constant drying rate is as follows when the heteropolyacid or salt thereof is, for example, silicotungstic acid.

Moisture content of impregnated body: y;

Supported catalyst mass (silica carrier mass+silicotungstic anhydride mass): C;

Water amount (hydration water of silicotungstic acid+water used to prepare aqueous heteropolyacid solution): x

When the silicotungstic acid after heat-drying is assumed to be anhydride, y is expressed as follows.

$$y = ( mass before heat - drying - mass after heat - drying ) / mass before heating - drying = [ ( C + x ) - C ] / ( C + x ) = x / ( C + x )$$

The drying rate (g_{H2O}/kg_{supcat}·min) is defined by dividing the difference (g) between the water amount x₀ before hot air drying and the water amount x₁ after drying for a predetermined time t, by the supported catalyst mass C (kg) and the drying time t (min).

Drying rate (g_{H2O}/kg_{supcat}·min)=(x₀−x₁)/(C×t)

At this time, y=x/(C+x) can be transformed into x=(C×y)/(1−y). Thus,

$$Drying rate ( gH 2 O ) / {kg}_{supcat} \cdot min ) = ( {x}_{0} - {x}_{1} ) / ( C \times t ) = [ ( C \times {y}_{0} ) / ( 1 - {y}_{0} ) - ( C \times {y}_{1} ) / ( 1 - {y}_{1} ) ] \text{} / ( C \times t ) = [ {y}_{0} / ( 1 - {y}_{0} ) - {y}_{1} / ( 1 - {y}_{1} ) ] / t$$

Note that the carrier catalyst mass C terms are canceled out in the denominator and numerator in the process of deriving the equation, and are thus not included in the drying rate equation.

The constant drying rate in the drying step is in the range of 5 to 300 g_{H2O}/kg_{supcat}·min, preferably in the range of 10 to 150 g_{H2O}/kg_{supcat}·min, and more preferably in the range of 15 to 50 g_{H2O}/kg_{supcat}·min. In another embodiment, the constant drying rate in the drying step is preferably in the range of 10 to 270 g_{H2O}/kg_{supcat}·min, and more preferably in the range of 15 to 240 g_{H2O}/kg_{supcat}·min. When the constant drying rate is less than 5 g_{H2O}/kg_{supcat}·min, it may not be possible to unevenly distribute the supported positions of heteropolyacid or salt thereof at the carrier surface. Conversely, when the constant drying rate exceeds 300 g_{H2O}/kg_{supcat}·min, the heteropolyacid or salt thereof may aggregate and sufficient catalyst performance may not be obtained.

As the drying method, general methods such as ambient pressure drying using hot air and reduced pressure drying can be adopted. From the viewpoint of cost and the number of operation steps, it is preferable to set the pressure in the drying step to ambient pressure (atmospheric pressure). The drying medium used in the drying step is preferably air, but may be an inert gas, such as nitrogen gas.

The type of drying device used in the drying step is not particularly limited. A method of contacting the impregnated body with a drying medium (such as hot air) as an aeration flow to dry the impregnated body is preferable. Examples of drying devices include, for example, band-type dryers and box-type dryers. It is preferable that the aeration flow not be circulated, but one pass (one-time flow) be adopted in the dryer. By using one pass, the drying medium with low humidity can always be brought into contact with the impregnated body (carrier on which the catalyst is supported), thereby increasing the constant drying rate.

The temperature of the drying medium is preferably in the range of 80 to 130° C., and more preferably in the range of 100 to 120° C. When the temperature of the drying medium is 80° C. or higher, the drying rate can be maintained at a certain value or higher, and the supported positions of the heteropolyacid or salt thereof can be unevenly distributed at the carrier surface. Conversely, when the temperature of the drying medium is 130° C. or lower, decomposition of the heteropolyacid or salt thereof can be suppressed.

When a heated flow of air or nitrogen gas is used as the drying medium, the wind speed is not particularly limited, but the linear velocity thereof is preferably in the range of 5 to 100 m/min, and more preferably in the range of 10 to 70 m/min. When the linear velocity is 5 m/min or more, the drying rate can be increased to effectively unevenly distribute the supported positions of the heteropolyacid or salt thereof at the carrier surface. On the other hand, if the linear velocity is 100 m/min or less, it is possible to suppress the catalyst (carrier) from whirling up during the drying step.

When air is used as the drying medium, the relative humidity thereof is preferably in the range of 0 to 60% RH, more preferably in the range of 0 to 40% RH, and further preferably in the range of 0 to 20% RH, based on the drying medium temperature at the time of entry into the drying device. When the humidity of the drying medium is 60% RH or less, the drying rate can be increased to effectively unevenly distribute the supported positions of the heteropolyacid or salt thereof at the carrier surface.

### [Ethyl Acetate Production]

In an embodiment, ethyl acetate can be obtained by reacting acetic acid and ethylene in a gas phase using a heteropolyacid or a salt thereof supported on a silica carrier as a solid acid catalyst. The acetic acid and ethylene are preferably diluted with an inert gas, such as nitrogen gas, from the standpoint of removing reaction heat. Specifically, a gas containing acetic acid and ethylene as raw materials is circulated in a vessel filled with a solid acid catalyst, and brought into contact with the solid acid catalyst to react them. It is preferable to add a small amount of water to the gas containing the raw materials from the viewpoint of maintaining catalyst activity, and in one embodiment, the reaction is carried out in the presence of water vapor. However, if excessive water is added, the amount of by-products, such as alcohols and ethers, may increase. The amount of water added is preferably 0.5 to 15 mol %, and more preferably 2 to 8 mol %, as a molar ratio of water to the sum of acetic acid, ethylene, and water.

The ratio of ethylene and acetic acid used as raw materials is not particularly limited, and as a molar ratio of ethylene and acetic acid, ethylene:acetic acid is preferably in the range of 1:1 to 40:1, more preferably in the range of 3:1 to 20:1, and further preferably in the range of 5:1 to 15:1.

The reaction temperature is preferably in the range of 50° C. to 300° C., and more preferably in the range of 140° C. to 250° C. The reaction pressure is preferably in the range of 0 PaG to 3 MPaG (gauge pressure), more preferably in the range of 0.1 MPaG to 2 MPaG (gauge pressure). In one embodiment, the reaction temperature is 150 to 170° C. and the reaction pressure is 0.1 to 2.0 MPaG.

Even though the SV of the gas (gas space velocity) containing the raw materials is not particularly limited, when it is excessively high, the raw materials will pass through without sufficient progress of the reaction, while if it is excessively low, problems such as low productivity may occur. The SV (volume of raw materials passing through 1 L of catalyst in 1 hour (L/L·h=h⁻¹)) is preferably 500 to 20,000 h⁻¹, more preferably 1,000 to 10,000 h⁻¹.

### EXAMPLES

The present invention will be described with reference to the following Examples and Comparative Examples, but the present invention is not limited to these Examples.

### [Bulk Density Measurement of Silica Carrier]

The bulk density of the silica carrier is measured by the following method.
1. Approximately 200 mL of the carrier is placed in a 1 L measuring cylinder.
2. Using a Kimtowel™ or the like as a cushioning material, the surrounding surface is tapped gently twenty times to ensure tight filling of the carrier.
3. 1 and 2 above are repeated multiple times.
4. When the volume of the carrier reaches approximately 1 L, the carrier is added in small increments, and operation 2 is repeated.
5. After measuring 1 L of the carrier, the mass is measured.
6. Operations 1 to 5 are performed three times in total, and the average of the mass values is taken as the bulk density (g/L).

### [Measurement of Saturated Water Absorption Capacity of Silica Carrier]

The saturated water absorption capacity of the silica carrier is measured at ambient temperature (23° C.) using the following measuring method.
1. Approximately 5 g of the carrier is weighed (W1 g) and placed in a 100 mL beaker.
2. Approximately 15 mL of pure water is added to the beaker so as to completely cover the carrier.
3. The beaker is allowed stand for 30 minutes.
4. The contents of the beaker are placed on a wire mesh with mesh openings which are smaller than the carrier, and the pure water is drained.
5. The water adhering to the surface of the carrier is removed by lightly pressing with a paper towel until the surface becomes dull.
6. The mass of the carrier which has absorbed water is measured (W2 g).
7. The saturated water absorption capacity of the carrier is calculated from the following formula.

Saturated water absorption capacity (volume of water absorbed (L)/apparent volume of carrier (L))=[(W2−W1)(g)/density of water at 23° C. (g/L)]×bulk density of carrier (g/L)/W1 (g)

### [Impregnation Ratio]

Impregnation ratio (%)=100×volume of aqueous heteropolyacid solution absorbed by carrier having apparent volume of 1 L/saturated water absorption capacity of carrier

### [Constant Drying Rate Calculation Method]

1. Approximately 5 g of the impregnated body is sampled and the moisture content thereof is measured with a heating balance.
2. The impregnated body is separately dried under predetermined conditions, and approximately 5 g of a supported catalyst (catalyst component+carrier) sample is taken within the constant rate drying period, and the moisture content thereof is measured with a heating balance.
3. The constant drying rate (g_{H2O}/kg_{supcat}·min) is calculated by dividing the amount of water removed by drying (g), which is obtained from the moisture contents in steps 1 and 2, by the drying time (min) and the supported catalyst mass (kg).

The drying conditions with a heating balance (heating dry moisture meter, model: MF-50, manufactured by A & D Company, Limited) are temperature: 200° C., end conditions: until the moisture content change becomes 0.05%/min.

The moisture content of the impregnated body is calculated by the formula described above. The impregnated body before heat-drying (before measuring the moisture content) contains hydration water of the heteropolyacid or salt thereof. The drying temperature in the heating balance is 200° C., and it is assumed that the hydration water is removed after heat-drying (after measuring the moisture content) and the heteropolyacid or salt thereof is an anhydride. Specifically, impregnated body mass before heat-drying=hydration water of heteropolyacid or salt thereof+silica carrier+water used to prepare aqueous heteropolyacid solution, and supported catalyst mass after heat-drying=anhydride of heteropolyacid or salt thereof+silica carrier.

### Example 1

### (Preparation of Catalyst A)

120 g of commercially available Keggin-type silicotungstic acid 26 hydrate (H₄SiW₁₂O₄₀·26H₂O; manufactured by Nippon Inorganic Colour & Chemical Co., Ltd.) was dissolved in 75.8 g (75.8 mL) of pure water to prepare 108 mL of an aqueous silicotungstic acid solution (95% by volume of the saturated water absorption capacity of the carrier, impregnation ratio 95%). Thereafter, the obtained aqueous solution was added to 0.3 L (134 g) of a commercially available silica carrier A (spherical shape, diameter: approximately 5 mm, bulk density: 451 g/L, saturated water absorption capacity: 379 g/L, BET specific surface area: 280 m²/g) and stirred well to impregnate the carrier. After air-drying for 1 hour, the impregnated body was dried with a ventilated box-type hot-air dryer (experimental ventilation rack-type dryer, model name: LABO-4CS, manufactured by Nagato Denki Mfg, Co., Ltd.) at a hot-air temperature of 100° C. and a wind speed of 13 m/min to obtain catalyst A. The constant drying rate was calculated by sampling 15 minutes after the start of drying. The value of the constant drying rate is shown in Table 1.

### Example 2

### (Preparation of Catalyst B)

An impregnated body was obtained in the same manner as Example 1 except that the amounts of the silicotungstic acid, pure water, and silica carrier were changed to 36.6 kg, 22.7 kg, and 90 L, respectively. The impregnated body was dried in the same manner as catalyst A, except that the hot-air temperature was changed to 100° C. and the wind speed was changed to 30 m/min, to obtain catalyst B. The value of the constant drying rate is shown in Table 1.

### Example 3

### (Preparation of Catalyst C)

Catalyst C was obtained by repeating the operations of Example 2 except that the wind speed of the hot air was changed to 60 m/min. The value of the constant drying rate is shown in Table 1.

### Example 4

### (Preparation of Catalyst D)

Catalyst D was obtained by repeating the operations of Example 3 except that the temperature of the hot air was changed to 120° C. The value of the constant drying rate is shown in Table 1.

### Example 5

### (Preparation of Catalyst E)

Catalyst E was obtained by repeating the operations of Example 1 except that the temperature of the hot air was changed to 130° C. and the wind speed was changed to 98 m/min. The value of the constant drying rate is shown in Table 1.

### Example 6

### (Preparation of Catalyst F)

120 g of commercially available Keggin-type silicotungstic acid 26 hydrate (H₄SiW₁₂O₄₀·26H₂O; manufactured by Nippon Inorganic Colour & Chemical Co., Ltd.) was dissolved in 73.3 g (73.3 mL) of pure water to prepare 105.5 mL of an aqueous silicotungstic acid solution (95% by volume of the saturated water absorption capacity of the carrier, impregnation ratio 95%). Thereafter, the obtained aqueous solution was added to 0.3 L (144 g) of a commercially available silica carrier B (spherical shape, diameter: approximately 5 mm, bulk density: 480 g/L, saturated water absorption capacity: 370 g/L, BET specific surface area: 147 m²/g) and stirred well to impregnate the carrier. Thereafter, the same operations as in Example 1 were repeated to obtain catalyst F. The value of the constant drying rate is shown in Table 1.

### Comparative Example 1

### (Preparation of Catalyst G)

Catalyst G was obtained by repeating the operations of Example 1, except that the dryer was changed to a natural convection box-type dryer (constant temperature dryer, model: DSR420DA, manufactured by Toyo Seisakusho Kaisha, Ltd.) set at a temperature of 100° C. The value of the constant drying rate is shown in Table 1.

### Comparative Example 2

### (Preparation of Catalyst H)

Catalyst H was obtained by repeating the operations of Example 1 except that the temperature of the hot air was changed to 50° C. and the wind speed was changed to 9 m/min. The value of the constant drying rate is shown in Table 1.

### Comparative Example 3

### (Preparation of Catalyst I)

Catalyst I was obtained by repeating the operations of Example 1 except that the impregnation ratio was changed to 70%. The value of the constant drying rate is shown in Table 1.

### Comparative Example 4

### (Preparation of Catalyst J)

0.3 L (144 g) of carrier B was impregnated with an aqueous silicotungstic acid solution in the same manner as in Example 6. After air-drying for 1 hour, drying was performed in the same manner as in Comparative Example 1 to obtain catalyst J. The value of the constant drying rate is shown in Table 1.

### [EPMA Analysis]

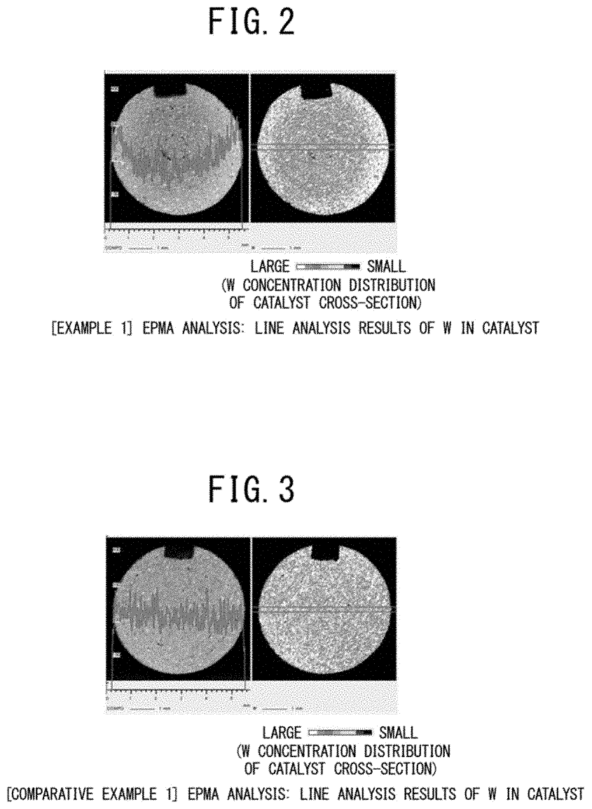

In order to confirm the supported locations of the active ingredient, the tungsten concentration distributions of the catalysts of Example 1 and Comparative Example 1 were measured by EPMA analysis. As a pretreatment of the measurement sample, the sample was split with a knife, and the cross section was roughly ground with #400, #1000, and #1500 abrasive paper in that order, a measurement surface was formed by finishing with #2000. The obtained results are shown in FIGS. 1 and 2. EPMA analysis was performed using the following equipment and conditions.
Apparatus: JXA-8530F (manufactured by JEOL Ltd.)
Acceleration voltage: 15 kV
WDS mapping (line analysis): WM line 3 ch (PET)
Irradiation current: 1×10⁻⁷ A
Measurement time: 50 ms
Beam diameter: 10 μm
Pixel size: 15 μm
Line analysis width: approximately 0.2 mm

### [Production of Ethyl Acetate]

A stainless-steel reaction tube having an inner diameter of 25 mm was filled with 40 mL of each of the catalysts obtained in the Examples and Comparative Examples described above, the pressure was increased to 0.75 MPaG, and subsequently, the temperature was raised to 155° C. After treating with a mixed gas of 85.5 mol % of nitrogen gas, 10.0 mol % of acetic acid, and 4.5 mol % of water under the condition of SV (volume of material passing through 1 L of catalyst in 1 hour (L/L·h=h⁻¹))=1500 h⁻¹ for 30 minutes, a mixed gas of 78.5 mol % of ethylene, 10 mol % of acetic acid, 4.5 mol % of water, and 7.0 mol % of nitrogen gas was introduced under the condition of SV=1500 h⁻¹ and reacted for 5 hours. The reaction was carried out by adjusting the reaction temperature so that the highest temperature among the 10 divided portions of the catalyst layer was 165.0° C. The gas passing through the reactor for 3 to 5 hours after the start of the reaction was condensed with cooling water and collected (hereinafter referred to as a “condensate”), and analyzed. In addition, the uncondensed gas remaining uncondensed (hereinafter referred to as “uncondensed gas”) was measured for the same amount of time as the condensate, and 100 mL of the gas was taken and analyzed. The obtained reaction results are shown in Table 1.

### [Condensate Analysis Method]

Using the internal standard method, 1 mL of 1,4-dioxane was added as an internal standard to 10 mL of the reaction solution, and 0.2 μL of the solution was injected and analyzed under the following conditions.
Gas chromatography apparatus: 7890B manufactured by Agilent Technologies
Column: capillary column DB-WAX (length: 30 m, inner diameter: 0.32 mm, film thickness: 0.5 μm)
Carrier gas: nitrogen gas (split ratio 200:1, column flow rate: 0.8 mL/min)
Temperature conditions: the detector temperature was 250° C., the vaporization chamber temperature was 200° C., the column temperature was maintained at 60° C. for 5 minutes from the start of analysis, and thereafter, the temperature was raised to 80° C. at a rate of 10° C./min, and after reaching 80° C., the temperature was raised to 200° C. at a rate of 30° C./min and maintained at 200° C. for 20 minutes.
Detector: FID (H₂ flow rate: 40 mL/min, air flow rate: 450 mL/min)

### [Uncondensed Gas Analysis Method]

Using the absolute calibration curve method, 100 mL of the uncondensed gas was sampled, the entire amount of this was flowed into a 1 mL gas sampler attached to the gas chromatography apparatus, and analysis was performed under the following conditions.

### 1. Ethyl Acetate

Gas chromatography apparatus: 7890A manufactured by Agilent Technologies
Column: Agilent J&W GC column DB-624
Carrier gas: He (flow rate: 1.7 mL/min)
Temperature conditions: the detector temperature was 230° C., the vaporization chamber temperature was 200° C., the column temperature was maintained at 40° C. for 3 minutes from the start of analysis, and then raised to 200° C. at a rate of 20° C./min.
Detector: FID (H₂ flow rate: 40 mL/min, air flow rate: 400 mL/min)

### 2. Butene

Gas chromatography apparatus: 7890A manufactured by Agilent Technologies
Column: Shimadzu GC GasPro (30 m), Agilent J&W GC column HP-1
Carrier gas: He (flow rate: 2.7 mL/min)
Temperature conditions: the detector temperature was 230° C., the vaporization chamber temperature was 200° C., and the column temperature was maintained at 40° C. for 3 minutes from the start of analysis, and then raised to 200° C. at a rate of 20° C./min.
Detector: FID (H₂ flow rate: 40 mL/min, air flow rate: 400 mL/min)

The tungsten concentration distribution of each catalyst by EPMA analysis is shown in FIG. 2 (Example 1) or FIG. 3 (Comparative Example 1). From FIGS. 2 and 3, it can be understood that by increasing the constant drying rate of the impregnated body, the supported positions of the heteropolyacid or salt thereof could be unevenly distributed at the outside of the carrier.

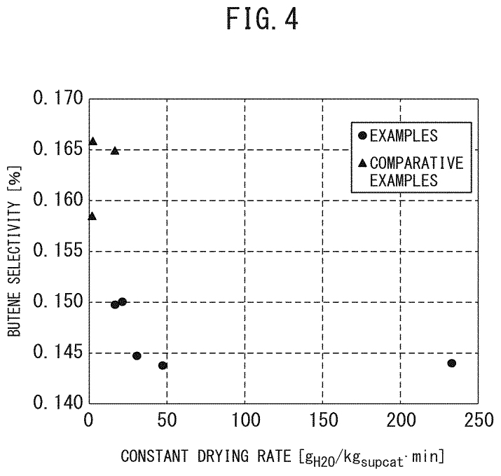

Catalyst performance results when producing ethyl acetate are shown in Table 1. When Examples 1 to 5 and Comparative Examples 1 and 2, which use the same carrier, are compared with each other, it can be understood that by increasing the constant drying rate, the space-time yield of ethyl acetate is increased and the selectivity of the by-product butene is decreased. In particular, as shown in FIG. 4, it can be understood that there is a correlation between the constant drying rate and butene selectivity. Since butene, which is one of the main by-products in this reaction, causes catalyst coking, smaller butene selectivity is desirable from the viewpoint of catalyst life. Though the catalysts differ in the butene selectivity by approximately several thousandths of a percent in the short-term evaluation in the present Examples, in light of the fact that tens of thousands of tons or more of ethyl acetate are produced annually during long-term operation in actual production, it is considered that this is a superior difference. Furthermore, when Comparative Example 3, in which the impregnation ratio is less than 80%, is compared with Examples 1 and 2, which use the same carrier and have a similar constant drying rate, it can be understood that the space-time yield of ethyl acetate is decreased and the butene selectivity is increased (deteriorated).

TABLE 1

| | | | Drying Conditions | | | | Catalyst Evaluation | | |
|---|---|---|---|---|---|---|---|---|---|
| | | | | | | Constant | | Ethyl acetate | |
| | | Impreg. | | Wind | | Drying Rate | | space-time | Butene |
| | | Ratio | Temp. | Speed | Drying | (g_{H2O}/ | | yield | selectivity |
| Example | Carrier | (%) | (° C.) | (m/min) | Method | kg_{supcat} · min) | Catalyst | (g/L · h) | (%) |
| Ex 1 | A | 95 | 100 | 13 | Vented hot- | 17 | A | 296 | 0.150 |
| | | | | | air dryer | | | | |
| Ex 2 | A | 95 | 100 | 30 | Vented hot- | 21 | B | 303 | 0.150 |
| | | | | | air dryer | | | | |
| Ex 3 | A | 95 | 100 | 60 | Vented hot- | 31 | C | 289 | 0.145 |
| | | | | | air dryer | | | | |
| Ex 4 | A | 95 | 120 | 60 | Vented hot- | 47 | D | 295 | 0.144 |
| | | | | | air dryer | | | | |
| Ex 5 | A | 95 | 130 | 98 | Vented hot- | 233 | E | 296 | 0.144 |
| | | | | | air dryer | | | | |
| Ex 6 | B | 95 | 100 | 13 | Vented hot- | 20 | F | 301 | 0.177 |
| | | | | | air dryer | | | | |
| Comp Ex 1 | A | 95 | 100 | — | Natural | 1.8 | G | 287 | 0.158 |
| | | | | | convection | | | | |
| | | | | | box dryer | | | | |
| Comp Ex 2 | A | 95 | 50 | 9 | Vented hot- | 2.3 | H | 292 | 0.166 |
| | | | | | air dryer | | | | |
| Comp Ex 3 | A | 70 | 100 | 13 | Vented hot- | 16 | I | 284 | 0.165 |
| | | | | | air dryer | | | | |
| Comp Ex 4 | B | 95 | 100 | — | Natural | 5.8 | J | 289 | 0.188 |
| | | | | | convection | | | | |
| | | | | | box dryer | | | | |

### INDUSTRIAL APPLICABILITY

The production method of the present invention can provide an ethyl acetate production catalyst having high productivity, wherein an active ingredient is present near the surface of a carrier, and high catalyst performance is exhibited, and has industrial applicability.

1. A method for producing an ethyl acetate production catalyst, comprising, in this order:
(1) an impregnation step of impregnating a silica carrier with an aqueous solution of a heteropolyacid or a salt thereof in an amount of 80 to 105% by volume of the saturated water absorption capacity of the carrier to form an impregnated body, and
(2) a drying step of drying the impregnated body at a constant drying rate of 5 to 300 g_{H2O}/kg_{supcat}·min.

2. The method for producing an ethyl acetate production catalyst according to claim 1, wherein the constant drying rate in the drying step is 10 to 150 g_{H2O}/kg_{supcat}·min.

3. The method for producing an ethyl acetate production catalyst according to claim 1, wherein the constant drying rate in the drying step is 15 to 50 g_{H2O}/kg_{supcat}·min.

4. The method for producing an ethyl acetate production catalyst according to claim 1, wherein a temperature of a drying medium used in the drying step is 80 to 130° C.

5. The method for producing an ethyl acetate production catalyst according to claim 1, wherein a drying medium in the drying step is air having a relative humidity of 0 to 60% RH, and the air is brought into contact with the impregnated body as an air flow to dry the impregnated body.

6. The method for producing an ethyl acetate production catalyst according to claim 1, wherein a pressure in the drying step is normal pressure.

7. A method for producing ethyl acetate using ethylene and acetic acid as raw materials, wherein a reaction is carried out in the presence of an ethyl acetate production catalyst produced by the method according to claim 1.
